(19) United States
(12) Patent Application Publication
(10) Pub. No.: US 2022/0313136 A1
(43) Pub. Date: Oct. 6, 2022

(54) PHYSIOLOGICAL INFORMATION PROCESSING APPARATUS, PHYSIOLOGICAL INFORMATION PROCESSING METHOD, AND NON-TRANSITORY COMPUTER READABLE STORAGE MEDIUM

(71) Applicant: NIHON KOHDEN CORPORATION, Tokyo (JP)

(72) Inventors: Kei KIMURA, Tokorozawa-shi (JP); Masafumi NAGASAWA, Tokorozawa-shi (JP)

(21) Appl. No.: 17/657,151

(22) Filed: Mar. 30, 2022

(30) Foreign Application Priority Data
Mar. 31, 2021 (JP) 2021-059671

(51) Int. Cl. A61B 5/33; A61B 5/358; A61B 5/00; A61B 5/28; A61B 5/352

(52) U.S. Cl. CPC A61B 5/33; A61B 5/358; A61B 5/746; A61B 5/28; A61B 5/352

(57) ABSTRACT

A physiological information processing instrument according to the presently disclosed subject matter can include a memory that stores instructions, a processor that executes the instructions stored in the memory to judge whether an electrocardiogram waveform of a subject person is of at least one of a ventricular pacing beat and a left bundle branch block beat, to perform an ST measurement by applying myocardial ischemia evaluation criteria to the electrocardiogram waveform that has been judged of at least one of a ventricular pacing beat and a left bundle branch block beat, and to analyze results of the ST measurement and outputs an analysis result and information relating to myocardial ischemia in the form of audible or visible information.

### CROSS-REFERENCE TO RELATED APPLICATIONS

The present application claims priority from Japanese Patent Application No. 2021-059671, filed on Mar. 31, 2021, the entire content of which is incorporated herein by reference.

### TECHNICAL FIELD

The present invention relates to a physiological information processing instrument, a physiological information processing method, and a physiological information processing instrument control program capable of performing myocardial ischemia monitoring on electrocardiogram waveforms of ventricular pacing or left bundle branch block.

### BACKGROUND ART

To monitor myocardial ischemia of myocardial infarction, angina pectoris, or the like, a deviation (ST value) of an ST segment (a segment between an S wave and a T wave) of an electrocardiogram waveform from the base line is measured. In general, an ST value is used as an index indicating occurrence/non-occurrence of myocardial ischemia in an electrocardiogram waveform that is obtained when a ventricle is contracted via a normal impulse conduction system. ST segments elevation and depression from the base line in an electrocardiogram waveform indicate occurrence of myocardial ischemia (refer to Japanese Patent No. 6,595,582).

However, an electrocardiogram waveform that is obtained in the case of ventricular pacing or left bundle branch block is much different in shape from an electrocardiogram waveform that is obtained in the case of general myocardial ischemia. An ST value that is obtained from a much different electrocardiogram waveform is much different from that obtained from an electrocardiogram waveform of a patient with general myocardial ischemia. Thus, in the case of ventricular pacing or left bundle branch block, it is difficult to perform myocardial ischaemia monitoring using only an ST value as in conventional procedures.

For the above-described reason, at present, myocardial ischemia monitoring using an ST value is not performed in the case of ventricular pacing or left bundle branch block.

### SUMMARY

### Technical Problem

In recent years, the Smith criteria and the Sgarbossa criteria have come to be known as criteria for ischemia evaluation in the case of ventricular pacing or tell bundle branch block.

Since even patients with ventricular pacing and patients of left bundle branch block may suffer myocardial ischemia, it is desired that myocardial ischemia monitoring be enabled.

However, since, for example, an ST value of a ventricular pacing beat is influenced by a pacing pulse that is output from a pacemaker, simply adding an ST value to monitoring targets may cause confusion about a displayed ST value. It is therefore preferable to deal with an ST value of a ventricular pacing beat differently rather than an ordinary ST value. Likewise, that is, as in an ST value of a ventricular pacing beat, it is preferable to deal with an ST value of a left bundle branch block beat differently rather than an ordinary ST value. It is known that an electrocardiogram waveform(s) of a ventricular pacing beat that is produced by setting leads in the right ventricle is similar to an electrocardiogram waveform of a left bundle branch block beat.

The present inventors thought that even in the case of ventricular pacing or left bundle branch block myocardial ischemia monitoring could be performed by applying the Smith criteria and the Sgarbossa criteria and making proper improvements in addition to using measurement values such as an ST value used in checking an electrocardiogram waveform conventionally.

The presently disclosed subject matter has been conceived to satisfy the above demand, and an object of the presently disclosed subject matter is therefore to provide a physiological information processing instrument, a physiological information processing method, and a physiological information processing instrument control program capable of performing myocardial ischemia monitoring even using ventricular pacing or left bundle branch block electrocardiogram waveforms.

### Solution to Problem

A physiological information processing instrument according to the presently disclosed subject matter for attaining the above object comprises a memory that stores instructions; and a processor that executes the instructions stored in the memory to judge whether an electrocardiogram waveform of a subject person is of at least one of a ventricular pacing beat and a left bundle branch block beat, to perform an ST measurement by applying myocardial ischemia evaluation criteria to the electrocardiogram waveform that has been judged of at least one of a ventricular pacing beat and a left bundle branch block beat, and to analyze results of the ST measurement and outputs an analysis result and information relating to myocardial ischemia in the form of audible or visible information.

A physiological information processing method according to the presently disclosed subject matter for attaining the above object comprises the steps of judging whether an electrocardiogram waveform of a subject person is of at least one of a ventricular pacing beat and a left bundle branch block beat; performing an ST measurement by applying myocardial ischemia evaluation criteria to the electrocardiogram waveform that has been judged of at least one of a ventricular pacing beat and a left bundle branch block beat; and analyzing results of the ST measurement and outputting an analysis result and information relating to myocardial ischemia in the form of audible or visible information.

A control program according to the presently disclosed subject matter for attaining the above object causes a computer that is to function as a physiological information processing instrument to function as a judging unit which judges whether an electrocardiogram waveform of a subject person is of at least one of a ventricular pacing beat and a left bundle branch block beat, an ST measuring unit which performs an ST measurement by applying myocardial ischemia evaluation criteria to the electrocardiogram waveform that has been judged of at least one of a ventricular pacing beat and a left bundle branch block beat; and an analyzing unit which analyzes results of the ST measurement and outputting an analysis result and information relating to myocardial ischemia in the form of audible or visible information.

### Advantageous Effect of Invention

The physiological information processing instrument, the physiological information processing method, and the physiological information processing instrument control program according to the presently disclosed subject matter enable myocardial ischemia monitoring using ventricular pacing or left bundle branch block electrocardiogram waveforms that has been difficult in the art.

### BRIEF DESCRIPTION OF DRAWINGS

FIG. 1 is a block diagram of a physiological information processing instrument according to an embodiment.

FIG. 2 is a main flowchart of the physiological information processing instrument according to the embodiment.

FIG. 3 is a subroutine flowchart of a step <generation of a representative waveform> in the flowchart shown in FIG. 2.

FIG. 4 is a subroutine flowchart of a step <ST measurement> in the flowchart shown in FIG. 2.

FIG. 5 is a subroutine flowchart of a step <judgment according to the Sgarbossa criteria> in the flowchart shown in FIG. 4.

FIG. 6 is a subroutine flowchart of a step <judgment according to the Smith criteria> in the flowchart shown in FIG. 4.

FIG. 7 is a subroutine flowchart of a step <judgment as to whether the notification conditions are satisfied> in the flowchart shown in FIG. 2.

FIG. 8 is a subroutine flowchart of a Specific Embodiment 2 version of the step <ST measurement> in the flowchart shown in FIG. 2.

FIG. 9 is a subroutine flowchart of a Specific Embodiment 2 or 3 version of a step <judgment according to the Sgarbossa criteria> in each of the flowcharts shown in FIGS. 8 and 12.

FIG. 10 is a subroutine flowchart of a Specific Embodiment 2 or 3 version of the step <judgment according to the Smith criteria> in each of the flowcharts shown in FIGS. 8 and 12.

FIG. 11 is a subroutine flowchart of a Specific Embodiment 2 version of the step <judgment as to whether the notification conditions are satisfied> in the flowchart shown in FIG. 2.

FIG. 12 is a subroutine flowchart of a Specific Embodiment 3 version of the step <ST measurement> in the flowchart shown in FIG. 2.

FIG. 13 is a subroutine flowchart of <left bundle branch block judgment> in the step <is each representative waveform of ventricular pacing or left bundle branch block?> in the flowchart of FIG. 12.

FIG. 14 is a subroutine flowchart of a Specific Embodiment 3 version of the step <judgement as to whether the notification conditions are satisfied> in the flowchart shown in FIG. 2.

FIG. 15 illustrates a normal electrocardiogram waveform.

FIG. 16 illustrates an electrocardiogram waveform that suggests myocardial ischemia.

FIG. 17 illustrates an rS-type waveform and a QS-type waveform that are used for judging whether a V1 lead is of left bundle branch block in the flowchart shown in FIG. 13.

FIG. 18 illustrates an R-type waveform that is used for judging whether a V6 lead is of left bundle branch block in the flowchart shown in FIG. 13.

FIG. 19 is a table illustrating an ST alarm setting range.

FIG. 20 is a table illustrating an STJ alarm setting range.

FIG. 21 is a table illustrating an STJ/QRS setting range.

FIG. 22 is a table illustrating setting ranges of Sgarbossa/Smith judgment threshold values.

FIG. 23 illustrates specific examples of an ST value and an ST recall waveform.

FIG. 24 is a table for description of display forms of an ST recall.

### DESCRIPTION OF EMBODIMENTS

Physiological information processing instruments according to an embodiment (i.e., Specific Embodiments 1-3) of the presently disclosed subject matter will be described below.

### Specific Embodiment 1

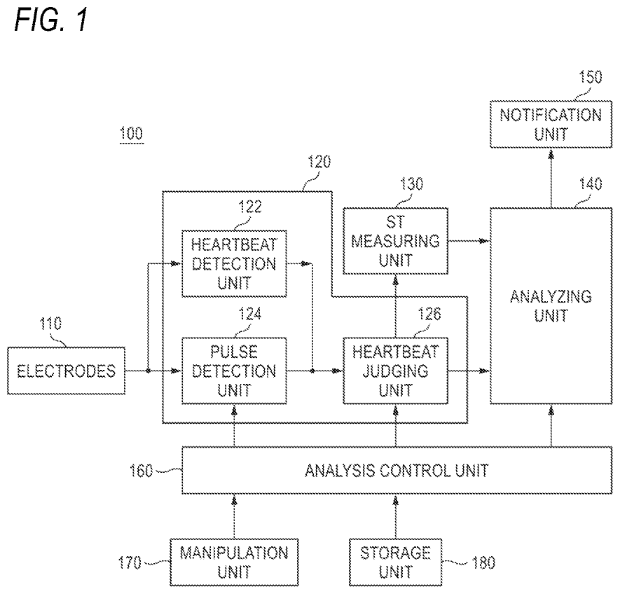

FIG. 1 is a block diagram of a physiological information processing instrument according to the embodiment. The configuration of the physiological information processing instrument shown in FIG. 1 is common to all of Specific Embodiments 1-3. The physiological information processing instrument 100 can include a judging unit 120, an ST measuring unit 130, and an analyzing unit 140.

The judging unit 120 receives an electrocardiogram waveform(s) from electrodes 110 that are attached to the body of a subject person and judges whether it is an electrocardiogram waveform of at least one of a ventricular pacing beat and a left bundle branch block beat.

The ST measuring unit 130 performs an ST measurement by applying myocardial ischemia evaluation criteria to the electrocardiogram waveform that has been subjected to the judgment by the judging unit 120. More specifically, the ST measuring unit 130 performs an ST measurement on an electrocardiogram waveform that has been subjected to the judgment by the judging unit 120 by applying, as its own myocardial ischemia evaluation criteria, judgement according to Smith criteria and the Sgarbossa criteria and measurement values relating to the shape of an electrocardiogram waveform that has been judged of a ventricular pacing beat or measurement values relating to the shape of an electrocardiogram waveform that has been judged of a left bundle branch block beat.

The analyzing unit 140 analyzes ST measurement results obtained by the ST measuring unit 130 and outputs an analysis result and information relating to myocardial ischemia, in the form of audible or visible information. An example of the manner of output of audible information is an output of an alarm sound and an example of the manner of output of visible information is display of a message More specifically, the analyzing unit 140 outputs an alarm if an STJ value exceeds a set alarm threshold value and outputs a message if an STJ value satisfies the Smith criteria and the Sgarbossa criteria even if it does not exceed the set alarm threshold value.

The judging unit 120 can include a heartbeat detection unit 122, a pulse detection unit 124, and a heartbeat Judging unit 126. The heartbeat detection unit 122 receives electrocardiogram waveforms from the electrodes 110 and detects heartbeats of a subject person. The pulse detection unit 124 detects presence/absence of a pacing pulse generated by a pacemaker that is attached to the subject person.

The heart beat judging unit 126 classifies part of electrocardiogram waveforms of the subject person as electrocardiogram waveforms of ventricular pacing beats or left bundle branch block beats or both on the basis of heartbeats of the subject person detected by the heartbeat detection unit 122 and presence/absence of a pacing pulse detected by the pulse detection unit 124, and generates a ventricular pacing beat group or a left bundle branch block beat group. Furthermore, the heartbeat judging unit 126 judges whether each electrocardiogram waveform of the subject person is an electrocardiogram waveform of at least one of a ventricular pacing beat and a left bundle branch block beat using a representative waveform generated from the ventricular pacing beat group or the left bundle branch block beat group.

The physiological information processing instrument 100 is also equipped with a notification unit 150. The notification unit 150 causes notification of the analysis result that is output from the analyzing unit 140 and the alarm or message relating to myocardial ischemia. The notification unit 150 displays, on a screen, the representative waveform generated by the judging unit 120 and measurement values relating to a shape of a electrocardiogram waveform as analysis results of the ST measurement performed by the ST measuring unit 130. The notification unit 150 also has a function of switching between electrocardiogram waveforms of a normal beat, a ventricular pacing beat, and a left bundle branch block beat, that is, displaying one of then on the screen selectively. The notification unit 151 further has a function of registering an electrocardiogram waveform of a normal beat, a ventricular pacing beat, or a left bundle branch block beat. Still further, the notification unit 150 displays an electrocardiogram waveform in such a manner that its frame line becomes deeper as its ST value becomes larger.

The physiological information processing instrument 100 is also equipped with an analysis control unit 160, a manipulation unit 170, and a storage unit (a memory) 180. The analysis control unit 160 controls the operations of the pulse detection unit 124, the heartbeat judging unit 126, and the analyzing unit 140 collectively. The manipulation unit 170 instructs the analysis control unit 160 as to various settings. The storage unit 180 is stored with control programs for the judging unit 120, the ST measuring unit 1311, the analyzing unit 140. Furthermore, the storage unit 180 stores information relating to electrocardiogram waveforms that are input or analyzed during processing of the judging unit 120, the ST measuring unit 130, the analyzing unit 140 and information to be displayed by the notification unit 150.

### (Operation of Physiological Information Processing Instrument)

The configuration of the physiological information processing instrument 100 according to the embodiment has been outlined above. Next, a description will be made of how the physiological information processing instrument 100 according to the embodiment operates.

The physiological information processing instrument 100 according to the embodiment operates to perform a physiological information processing method of the physiological information processing instrument 100. The physiological information processing method is a physiological information processing method that makes it possible to perform myocardial ischemia monitoring using even an electrocardiogram waveform of ventricular pacing or left bundle branch block, and includes the steps of judging whether an electrocardiogram waveform of a subject person is of at least one of a ventricular pacing beat and a left bundle branch block beat, performing an ST measurement by applying myocardial ischemia evaluation criteria to the electrocardiogram waveform that has been judged of at least one of a ventricular pacing beat and a left bundle branch block beat, and analyzing results of the ST measurement and outputting an analysis result and an alarm or message relating to myocardial ischemia.

The operation of the physiological information processing instrument 100 according to the embodiment is controlled by a computer (a processor) that is part of the physiological information processing instrument 100. A control program for controlling the operation of the physiological information processing instrument 100 is stored in the storage unit 180 shown in FIG. 1. The storage unit 180 may be a memory or non-transitory computer-readable storage medium that includes instructions performed by the processor. The control program of the physiological information processing instrument 100 is a control program of the physiological information processing instrument 100 capable of myocardial ischemia monitoring using even an electrocardiogram waveform of ventricular pacing or left bundle branch block and causes a computer that is to function as the physiological information processing instrument 100 to function as the judging unit 120 which judges whether an electrocardiogram waveform of a subject person is of at least one of a ventricular pacing beat and a left bundle branch block beat, the ST measuring unit 130 which performs an ST measurement by applying myocardial ischemia evaluation criteria to the electrocardiogram waveform that has been judged of at least one of a ventricular pacing beat and a left bundle branch block beat, and the analyzing unit 140 which analyzes results of the ST measurement and outputting an analysis result and an alarm or message relating to myocardial ischemia.

### (Main Flowchart)

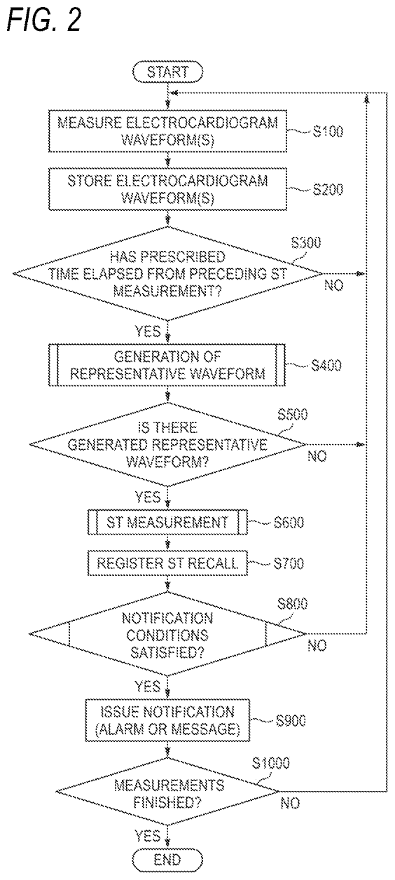

FIG. 2 is a main flowchart of the physiological information processing instrument according to the embodiment. The main flowchart shown in FIG. 2 is followed by the computer that is part of the physiological information processing instrument 100. The main flowchart shown in FIG. 2 is common to Specific Embodiments 1-3.

First, the judging unit 120 receives an electrocardiogram waveform(s) of a subject person from the electrodes 110 and measures it. Methods using three electrodes, six electrodes, or 10 electrodes are mainly employed as a method for monitoring electrocardiogram waveforms. The judging unit 120 measures a 1-lead electrocardiogram waveform in the case of three electrodes, 8-lead electrocardiogram waveforms in the case of six electrodes, and 12-lead electrocardiogram waveforms in the case of 10 electrodes (S100). In Specific Embodiment 1, a 1-lead electrocardiogram waveform is measured because use of three electrodes is assumed.

Then the storage unit 180 stores the electrocardiogram waveform(s). The storage unit 180 stores a 1-lead electrocardiogram waveform (one electrocardiogram waveform) in the case of three electrodes, 8-lead electrocardiogram waveforms (8 electrocardiogram waveforms) in the case of six electrodes, and 12-lead electrocardiogram waveforms (12 electrocardiogram waveforms) in the case of 10 electrodes (S200). In Specific Embodiment 1, a 1-lead electrocardiogram waveform is stored because use of three electrodes is assumed.

Then the judging unit 120 judges whether a prescribed time has elapsed from the preceding ST measurement that was performed by the ST measuring unit 130 (S300). More specifically, the ST measuring unit 130 performs an ST measurement on an electrocardiogram waveform by applying, as its myocardial ischaemia evaluation criteria, judgement according to Smith criteria and the Sgarbossa criteria and measurement values relating to the shape of an electrocardiogram waveform that has been judged of a ventricular pacing beat or measurement values relating to the shape of an electrocardiogram waveform that has been judged of a left bundle branch block beat. Thus, the judging unit 120 judges whether a prescribed time has elapsed from such an ST measurement. The details of the ST measurement will be described later.

If the prescribed time has not elapsed from the preceding ST measurement (S300: no), the judging unit 120 executes step S100. On the other hand, if the prescribed time has elapsed from the preceding ST measurement (S300: yes), the judging unit 120 generates a representative waveform from electrocardiogram waveforms stored in the storage unit 180. More specifically, the judging unit 120 classifies each of the electrocardiogram waveforms of the subject person stored in the storage unit 180 as an electrocardiogram waveform of at least one of a ventricular pacing beat and a left bundle branch block beat, generates a ventricular pacing beat group or a left bundle branch block beat group, and generates a representative waveform from the ventricular pacing beat group or the left bundle branch block beat group. There may occur an event that a representative waveform cannot be generated if each electrocardiogram waveform stored in the storage unit 180 contain much noise or the electrocardiogram waveforms were not grouped properly. The details of how to generate an electrocardiogram waveform will be described later.

The judging unit 120 judges whether a generated representative waveform exists (S500). If no generated representative waveform exists (S500; no), the judging unit 120 executes step S100. On the other hand, if a generated representative waveform exists (S500: yes), the ST measuring unit 130 performs an ST measurement (S600). More specifically, where the number of electrodes attached to the subject person is three (i.e., 3-electrode case), a judgment according to the Smith criteria and the Sgarbossa criteria is made by judging whether an electrocardiogram waveform that has been judged of a ventricular pacing beat satisfies one kind of a set of Smith criteria and Sgarbossa criteria.

Where the number of electrodes attached to the subject person is six or ten (i.e., 6-electrode case or 10-electrode case), a judgment according to the Smith criteria and the Sgarbossa criteria is made on the basis of a score, determined according to a set of Smith criteria and Sgarbossa criteria that is different than employed in the 3-electrode case, of an electrocardiogram waveform that has been judged of a ventricular pacing beat. In the 10-electrode case, a judgment according to the Smith criteria and the Sgarbossa criteria is made on the basis of a score, determined according to the same set of Smith criteria and Sgarbossa criteria as employed in the 6-electrode case, of an electrocardiogram waveform that has been judged of a left bundle branch block beat.

Where the number of electrodes attached to the subject person is six or ten (i.e., 6-electrode case or 10-electrode case), a judgment according to the Smith criteria and the Sgarbossa criteria is made by determining a score of an electrocardiogram waveform of a ventricular pacing beat or a left bundle branch block beat of each lead according to the Smith criteria and the Sgarbossa criteria and calculating a total score of all leads.

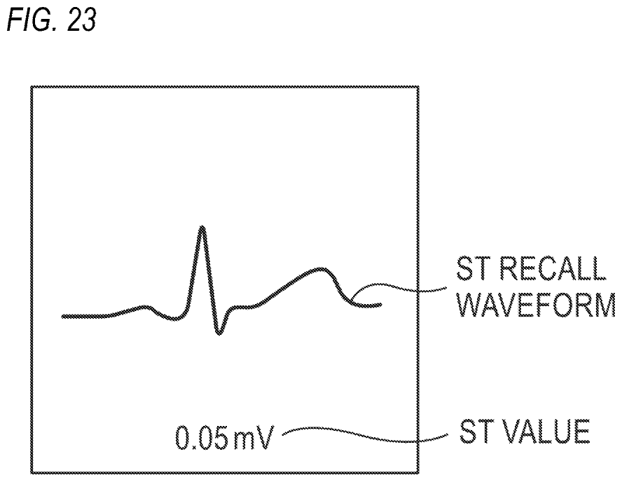

The analyzing unit 140 receives a judgment result of the ST measuring unit 130 and registers an ST recall (S700). More specifically, an ST value and an ST recall waveform as shown in FIG. 23 are registered. The analyzing unit 140 judges whether the registered ST recall satisfies notification conditions (S800). If the registered ST recall does not satisfy the notification conditions (S800: no), the judging unit 120 executes step S100. On the other hand, if the registered ST recall satisfies the notification conditions (S800: yes), the analyzing unit 140 outputs a notice (alarm or message) to the notification unit 150 (S900). Receiving this notice, the notification unit 150 announces an analysis result that is output from the analyzing unit 140 and information relating to myocardial ischemia, in the form of audible or visible information. The details of processing for judging whether the notification conditions are satisfied will be described later.

The analysis control unit 160 judges whether measurements have finished (S1000). If measurements have not finished (S1000: no), the judging unit 120 executes step S100. On the other hand, if measurements have finished (S1000: yes), the measurement process is finished.

The process of the main flowchart to be followed by the physiological information processing instrument according to Specific Embodiment 1 has been outlined above. Next, subroutine flowcharts of step S400 <generation of a representative waveform>, step S600 <ST measurement>, and step S800 <notification conditions satisfied?> of the main flowchart shown in FIG. 2 will be described.

### (Subroutine Flowcharts)

### <Generation of a Representative Waveform>

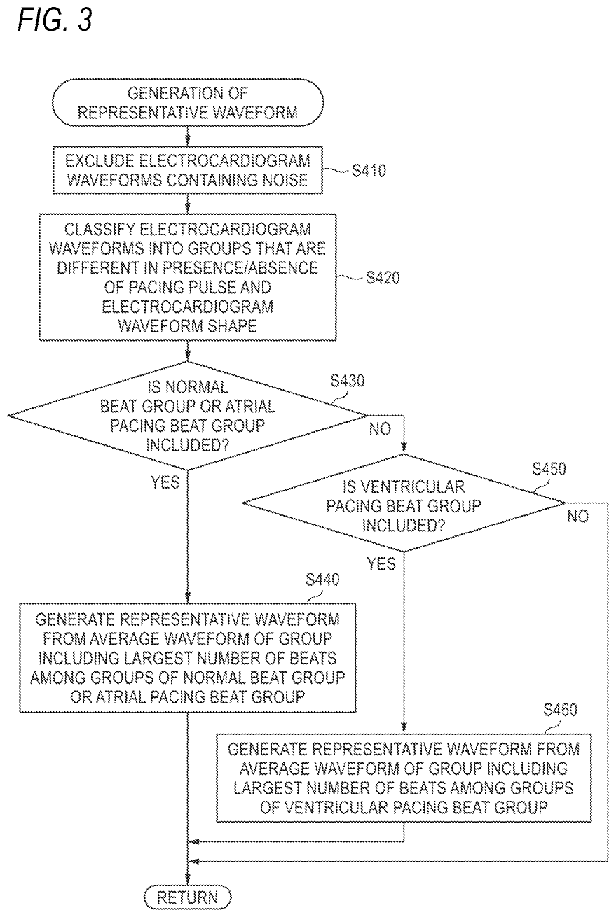

FIG. 3 is a subroutine flowchart of the step <generation of a representative waveform> in the flowchart shown in FIG. 2. The subroutine flowchart shown in FIG. 3 is common to Specific Embodiments 1-3.

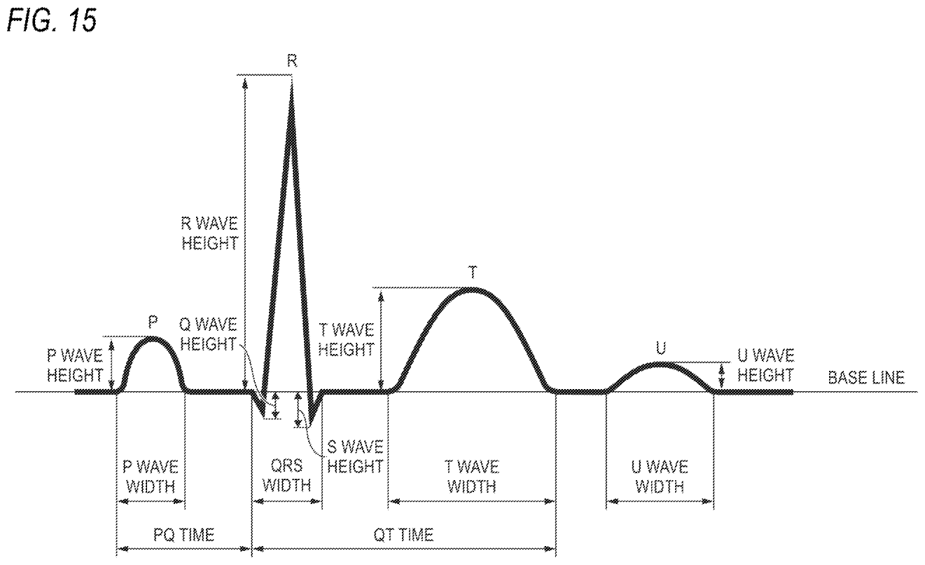

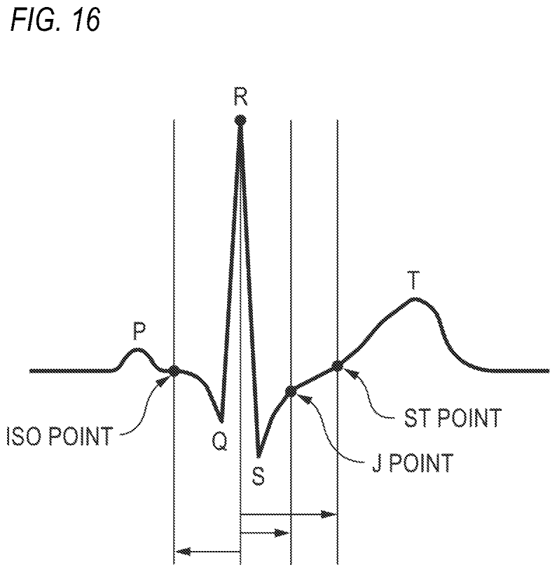

The heartbeat judging unit 126 monitors electrocardiogram waveforms that are input from the heartbeat detection unit 122 and excludes ones containing noise (S410). Whether an electrocardiogram waveform contains noise is judged by monitoring differences in shape between a standard electrocardiogram waveform shown in FIG. 15 and an input electrocardiogram waveform and whether feature values of individual portions are within allowable ranges. For example, an input electrocardiogram waveform is excluded as containing noise if its individual points such as an ISO point, a J point, and an ST point (see FIG. 16) deviate from allowable ranges to a large extent. Whether noise is contained can be judged using a technique commonly used in the art. The individual points such as the ISO point, J point, and ST point can be set in a desired manner by the manipulation unit 170. In FIG. 15, definitions are as follows:

R PEAK: HEIGHT OF R WAVE,

ST SEGMENT: SEGMENT BETWEEN S WAVE AND T WAVE,

ST VALUE: DEVIATION OF ST SEGMENT FROM BASE LINE,

QRS START PORTION: POINT WHERE Q WAVES STARTS,

QRS MAIN PEAK: HIGHEST POINT OF Q WAVE, R WAVE, AND S WAVE,

QRS WIDTH: WIDTH FROM START OF Q WAVE TO END OF S WAVE.

In FIG. 16, definitions are as follows:

ISO POINT: FLAT POINT OF QRS START PORTION,

MEASUREMENT REFERENCE POINT: ISO POINT OR J POINT,

STJ VALUE: POTENTIAL AT J POINT,

ST60 VALUE: POTENTIAL AT POINT THAT IS 60 ms AFTER J POINT,

QRS MAIN PEAK AMPLITUDE: HEIGHT OF QRS MAIN PEAK FROM, MEASUREMENT REFERENCE POINT,

STJ CHANGE: CHANGE OF STJ VALUE WITH RESPECT TO ISO POINT,

STJ CHANGE AMOUNT: CHANGE AMOUNT OF STJ VALUE WITH RESPECT TO POTENTIAL AT ISO POINT.

Next, the heartbeat judging unit 126 classifies electrocardiogram waveforms into groups that are different in presence/absence of a pacing pulse and electrocardiogram waveform shape (S420). Presence/absence of a pacing pulse can be judged on the basis of an output of the pulse detection unit 124. If a pacemaker is attached to the subject person, a pacing pulse may be included in an electrocardiogram waveform. If a pacing pulse is detected by the pulse detection unit 124, that fact is output to the heartbeat judging unit 126 and hence the heartbeat judging unit 126 can recognize whether the electrocardiogram waveform detected by the heartbeat detection unit 122 contains a pacing pulse. Thus, the heartbeat judging unit 126 can classify received electrocardiogram waveforms into groups that are different in presence/absence of a pacing pulse and electrocardiogram waveform shape. The classification into groups that are different in electrocardiogram waveform shape may be performed either using similarities between the shapes themselves of electrocardiogram waveforms (see FIGS. 15 and 16) or through measurement of feature values of individual portions of each electrocardiogram waveform. An example of the classification performed at this step is classification into a normal beat group, an atrial pacing beat group, and a ventricular pacing beat group. The electrocardiogram waveforms belonging to each of the normal beat group, the atrial pacing beat group, and the ventricular pacing beat group are further classified into groups either using similarities between the shapes themselves of the electrocardiogram waveforms or feature values of individual portions of each electrocardiogram waveform. For example, the electrocardiogram waveforms belonging to the normal beat group are further classified into groups either using similarities between the shapes themselves of the electrocardiogram waveforms or feature values of individual portions of each electrocardiogram waveform. The same is true of the atrial pacing beat group and the ventricular pacing beat group.

Subsequently, the heartbeat judging unit 126 judges whether the groups of electrocardiogram waveforms obtained at step S420 include a normal beat group or an atrial pacing beat group (S430). If a normal beat group or an atrial pacing beat group is included (S430: yes), the heartbeat judging unit 126 generates a representative waveform from an average waveform of a group including a largest number of beats among the groups of the normal beat group or the atrial pacing beat group (S440). Since each of the normal beat group and the atrial pacing beat group is classified into groups using similarities between the shapes themselves of electrocardiogram waveforms or feature values of individual portions of each electrocardiogram waveform, in the case of the normal beat group an average waveform of a group including a largest number of beats among the groups of the normal beat group (i.e., an averaged waveform of the electrocardiogram waveforms belonging to the group) is generated and employed as a representative waveform of the normal waveforms. The same is true of the atrial pacing beat group.

On the other hand, if neither a normal beat group nor an atrial pacing beat group is included (S430: no), the heartbeat judging unit 126 judges whether a ventricular pacing group is included (S450). If a ventricular pacing group is included (S450: yes), the heart beat judging unit 126 generates a representative waveform from an average waveform of a group including a largest number of beats among the groups of the ventricular pacing beat group (S460) If there exists no ventricular pacing beat group (S450: no), the step “generation of a representative waveform” is finished.

### <ST Measurement>

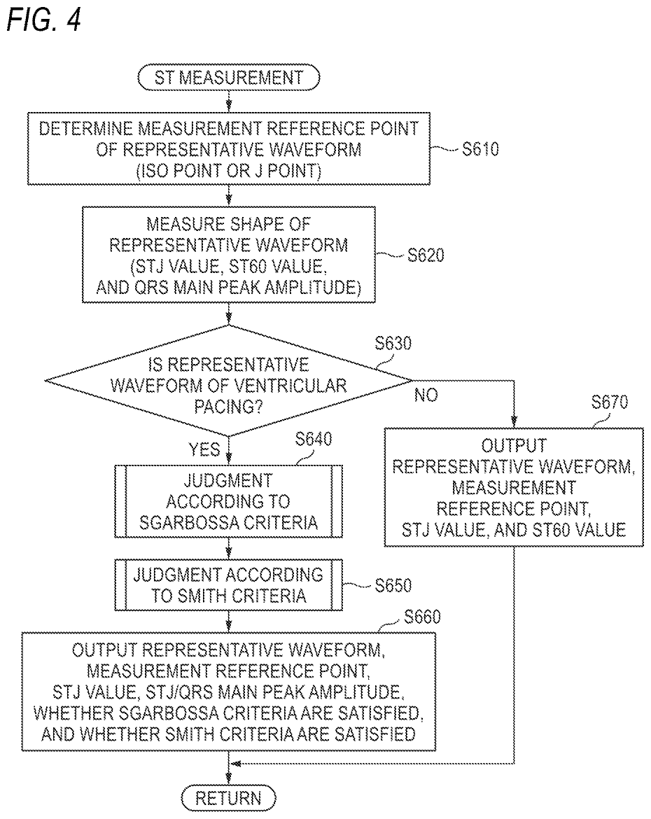

FIG. 4 is a subroutine flowchart of the step <ST measurement> in the flowchart shown in FIG. 2. The subroutine flowchart shown in FIG. 4 is applied to only Specific Embodiment 1 which assumes use of three electrodes.

The ST measuring unit 130 determines a measurement reference point of the representative waveform generated by the subroutine flowchart of <generation of a representative waveform> (see FIG. 3) (610). The measurement reference point of the representative waveform is an ISO point or a J point indicated in the electrocardiogram waveform shown in FIG. 16. The ISO point is a potential at a flat point of a QRS start portion (i.e., a point where a Q wave starts) and the J point is a potential at an inflection point of a curve that goes from an S wave to a T wave.

Then the ST measuring unit 130 measures a shape of the representative waveform (S620). A shape of the representative waveform is not determined qualitatively, that is, it is represented by feature values determined quantitatively at individual portions of the representative waveform such as an STJ value, an ST60 value, and a QRS main peak amplitude indicated in the electrocardiogram waveform shown in FIGS. 15 and 16. The STJ value is a potential at the J point, the ST60 value is a potential at a point that is 60 msec after the J point, and the QRS main peak amplitude is a potential at a highest point of the Q wave, the R wave, and the S wave with respect to a base line.

Then the ST measuring unit 130 judges whether the representative waveform is of ventricular pacing (S630). If the representative waveform is of ventricular pacing (S630: yes), the ST measuring unit 130 makes judgment according to Sgarbossa criteria (S640) and judgment according to Smith criteria (S650) and outputs the representative waveform, the measurement reference points, the STJ value, the ratio (STJ change amount)/(QRS main peak amplitude), whether the Sgarbossa criteria are satisfied, and whether the Smith criteria are satisfied to the analyzing unit 140 (S660). The judgment according to the Sgarbossa criteria and the judgment according to Smith criteria will be described later.

On the other hand, if the representative waveform is not of ventricular pacing (S630: no), the ST measuring unit 130 outputs the representative waveform, the measurement reference point, the STJ value, and the ST60 value to the analyzing unit 140 (S670)

### <Judgment According to Sgarbossa Criteria>

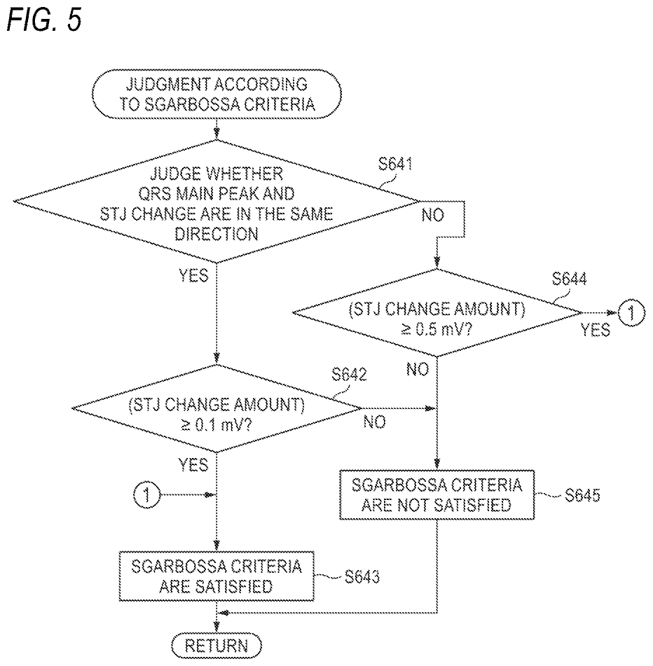

FIG. 5 is a subroutine flowchart of the step <judgment according to the Sgarbossa criteria> in the flowchart shown in FIG. 4. The subroutine flowchart shown in FIG. 5 is applied to only Specific Embodiment 1 which assumes three electrodes.

The analyzing unit 140 judges whether the QRS main peak and the STJ change of the representative waveform are in the same direction as shown in FIGS. 15 and 16 (S641). Since the QRS main peak is a highest point of the Q wave, the R wave, and the S wave and the STJ change is a change of the STJ value (i.e., a potential at the J point) with respect to the ISO point, if the representative waveform is an electrocardiogram waveform as shown in FIGS. 15 and 16 the QRS main peak and the STJ change of the representative waveform are both in the positive direction and hence are judged to be in the same direction. Like Sgarbossa/Smith judgment threshold values shown in FIG. 22, a setting range of the STJ change that is in the same direction as the QRS main peak can be set to a prescribed range by the manipulation unit 170. The setting range is from 0.10 mV to 1.00 mV (initial value: 0.1 mV, interval: 0.01 mV). Alternatively, a setting range can be set in the form of a distance in mm on a graph of the electrocardiogram waveform rather than voltages. In this case, a setting range is set so as to be from 1.0 mm to 10.0 mm (initial value: 10 mm, interval: 0.1 mm). A setting range of the STJ change that is in the opposite direction to the QRS main peak can be set to a prescribed range by the manipulation unit 170 like a Sgarbossa-Smith judgment threshold value shown in FIG. 22. The setting range is from 0.10 mV to 1.0 mV (initial value: 0.5 mV, interval: 0.01 mV). Alternatively, a setting range can be set in the form of a distance in mm on a graph of the electrocardiogram waveform rather than voltages. In this case, a setting range is set so as to be from 1.0 mm to 10.0 mm (initial value: 5.0 mm, interval: 0.1 mm).

If the QRS main peak and the STJ change of the representative waveform are in the same direction (S641: yes), it is then judged whether the STJ change amount is larger than or equal to 0.1 mV (S642). If the STJ change amount is larger than or equal to 0.1 mV (S642: yes), the representative waveform is judged to satisfy the Sgarbossa criteria (643).

If the QRS main peak and the STJ change of the representative waveform are not in the same direction (S641: no), it is then judged whether the STJ change amount is larger than or equal to 0.5 n (S644). If the STJ change amount is larger than or equal to 0.5 mV, the representative waveform is judged to satisfy the Sgarbossa criteria (S643).

If the STJ change amount is judged smaller than 0.1 mV at step 3642 (S642: no) or judged smaller than 0.5 mV at step S644 (S644: no), the representative waveform is judged not to satisfy the Sgarbossa criteria (S645).

### <Judgment According to Smith Criteria>

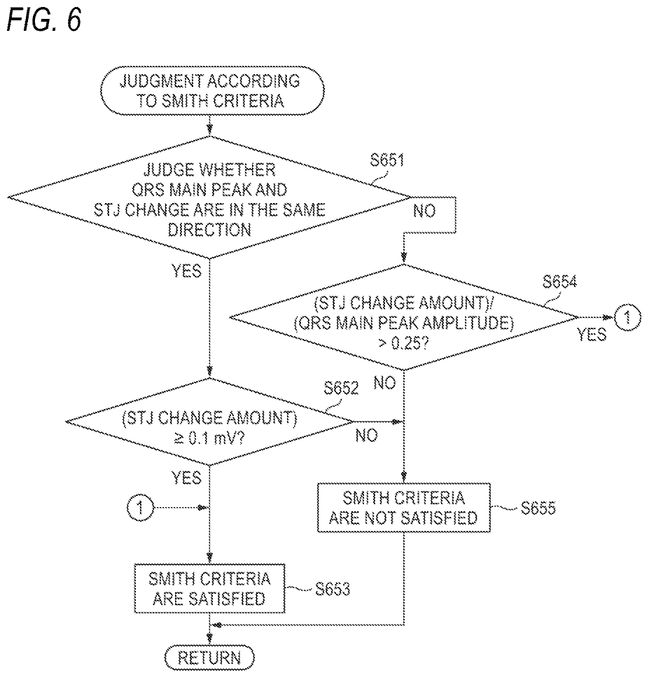

FIG. 6 is a subroutine flowchart of the step <judgment according to the Smith criteria in the flowchart shown in FIG. 4. The subroutine flowchart shown in FIG. 6 is applied to only Specific Embodiment 1 which assumes three electrodes.

The analyzing unit 140 judges whether the QRS main peak and the STJ change of the representative waveform are in the same direction as shown in FIGS. 15 and 16 (S651). Since the QRS main peak is a highest point of the Q wave, the R wave, and the S wave and the STJ change is a change of the STJ value (i.e., a potential at the J point) with respect to the ISO point, if the representative waveform is an electrocardiogram waveform as shown in FIGS. 15 and 16 the QRS main peak and the STJ change of the representative waveform are both in the positive direction and hence are judged to be in the same direction. Like the Sgarbossa/Smith judgment threshold value shown in FIG. 22, a setting range of the STJ change that, is in the same direction as the QRS main peak can be set to a prescribed range by the manipulation unit 170 (see FIG. 1) in the same manner as described above.

If the QRS main peak and the STJ change of the representative waveform are in the same direction (S651: yes), it is then judged whether the STJ change amount is larger than or equal to 0.1 mV (S652) If the STJ change amount is larger than or equal to 0.1 mV (S652: yes), the representative waveform is judged to satisfy the Smith criteria (S653).

If the QRS main peak and the STJ change of the representative waveform are not in the same direction (S651: no), it is then judged whether the ratio (STJ change amount)/(QRS main peak amplitude) is larger than 0.25 (S654). If the ratio (STJ change amount)/(QRS main peak amplitude) is larger than 0.25, the representative waveform is judged to satisfy the Smith criteria (S653).

If the STJ change amount is judged smaller than 0.1 mV at step S652 (S652: no) or the ratio (STJ change amount)/(QRS main peak amplitude) is judged smaller than or equal to 0.25 at step S654 (S654: no), the representative waveform is judged not to satisfy the Smith criteria (8655).

### <Judgment as to Whether the Notification Conditions are Satisfied>

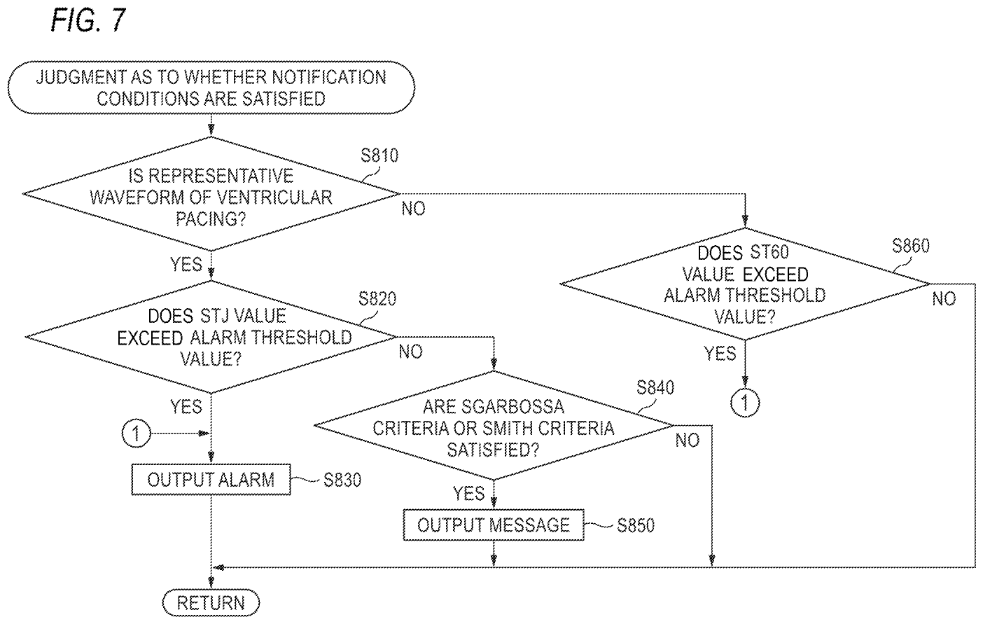

FIG. 7 is a subroutine flowchart of the step <judgment as to whether the notification conditions are satisfied> in the flowchart shown in FIG. 2. The subroutine flowchart shown in FIG. 7 is applied to only Specific Embodiment 1 which assumes three electrodes.

The ST measuring unit 130 judges whether the representative waveform generated in the subroutine flowchart of the step <generation of a representative waveform> (see FIG. 3 is of ventricular pacing (S810). If the representative waveform is of ventricular pacing (S810: yes), then the analyzing unit 140 judges whether the STJ value exceeds an alarm threshold value (S820). As shown in FIG. 20, an upper limit value and a lower limit value of an alarm relating to the STJ value can each be set in a prescribed range by the manipulation unit 170 (see FIG. 1). The interval of a setting range is 0.01 mV and its upper limit value is from −1.99 mV to +2.00 mV and its lower limit value is from −2.00 mV to +1.99 mV. The setting of the upper limit value and the lower limit value can be made off. Alternatively, a setting range can be set in the form of a distance in mm on a graph of the electrocardiogram waveform rather than voltages. In this case, the interval of a setting range is 0.1 mm and its upper limit value is from −19.99 mm to +20.0 mm and its lower limit value is from −20.0 mm to +19.99 mm. The setting of the upper limit value and the lower limit value can be made off.

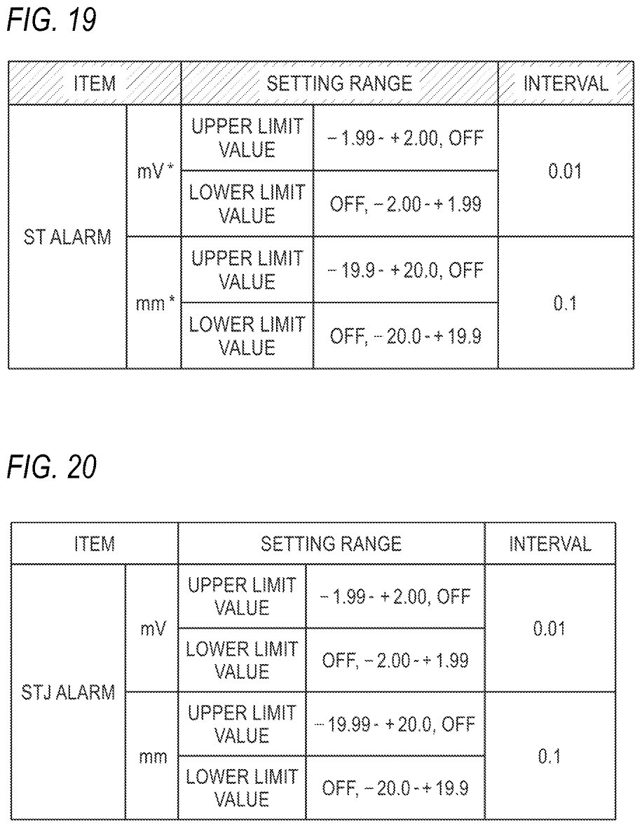

If the STJ value exceeds the alarm threshold value (S820: yes), the notification unit 150 outputs an alarm (S830). On the other hand, if the representative waveform is not of ventricular pacing (S810: no), the analyzing unit 140 then judges whether the ST60 value exceeds an alarm threshold value (S860). As shown in FIG. 19, an upper limit value and a lower limit value of an alarm relating to the ST60 value can each be set in a prescribed range by the manipulation unit 170 (see FIG. 1). The interval of a setting range is 0.01 mV and its upper limit value is from −0.99 mV to +2.00 mV and its lower limit value is from −2.00 mV to +1.99 mV. The setting of the upper limit value and the lower limit value can be made off. Alternatively, a setting range can be set in the form of a distance in mm on a graph of the electrocardiogram waveform rather than voltages. In this case, the interval of a setting range is 0.1 mm and its upper limit value is from −19.9 mm to +20.0 mm and its lower limit value is from −200 mm to +19.9 mm. The setting of the upper limit value and the lower limit value can be made off.

If it is judged that the ST60 value exceeds the alarm threshold value (S860: yes), the notification unit 150 outputs an alarm (S830). On the other hand, if the STJ value does not exceed the alarm threshold value (S820: no) then the analyzing unit 140 judges whether the Sgarbossa criteria or the Smith criteria are satisfied (S840). If the Sgarbossa criteria or the Smith criteria are satisfied (S840: yes), the notification unit 150 outputs a message (S850).

If it is judged at step S860 that the ST60 value does not exceed the alarm threshold value (S860: no) or it is judged at step S840 that neither the Sgarbossa criteria nor the Smith criteria are satisfied (S840: no), the process is finished without issuing any notice.

How the physiological information processing instrument 100 according to Specific Embodiment 1 operates according to each of the subroutine flowcharts has been outlined above. In summary, the physiological information processing instrument 100 according to Specific Embodiment 1 operates as follows. Electrocardiogram waveforms of a subject person are measured and stored until lapse of a prescribed time. The stored electrocardiogram waveforms are classified into groups of at least normal beats, atrial pacing beats, and ventricular pacing beats, and a representative waveform is generated. An ST measurement is performed on the generated electrocardiogram waveform and an ST recall is registered. An alarm or a message is issued if notification conditions are satisfied. This process makes it possible to perform myocardial ischemia monitoring on electrocardiogram waveforms of ventricular pacing that has been difficult to perform in the art.

### Specific Embodiment 2

### (Configuration of Physiological Information Processing Instrument)

The configuration of the physiological information processing instrument according to this Specific Embodiment is the same as shown in FIG. 1 (block diagram).

### (Operation of Physiological Information Processing Instrument)

The physiological information processing instrument 100 according to this Specific Embodiment operates, in outline, according to the same procedure as shown in the main flowchart of FIG. 2. However, in this Specific Embodiment, since the number of electrodes attached to a subject person is six that is different than in Specific Embodiment 1 and the number of electrocardiogram waveforms obtained is eight, in the main flowchart of FIG. 2 8-lead electrocardiogram waveforms are measured at step S100 and 8-lead electrocardiogram waveforms are stored at step S200. And subroutine flowcharts of step S600 <ST measurement> and step S800 <notification conditions satisfied?> are somewhat different than in Specific Embodiment 1. The procedures of these subroutine flowcharts will be described below.

### <ST Measurement>

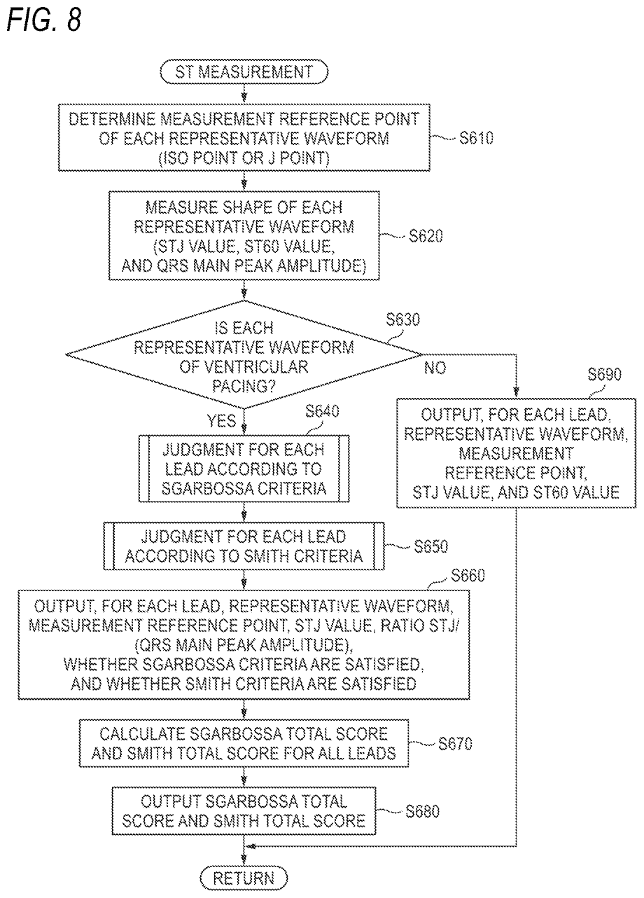

FIG. 8 is a subroutine flowchart of a Specific Embodiment 2 version of the step <ST measurement> in the flowchart of FIG. 2. The subroutine flowchart shown in FIG. 8 is applied to only Specific Embodiment 2 which employs six electrodes.

The ST measuring unit 130 determines a measurement reference point of each representative waveform that was generated in the subroutine flowchart <generation of a representative waveform> (see FIG. 3) (S610) In this Specific Embodiment, representative waveforms are generated for the 8-lead electrocardiogram waveforms. The measurement reference point of each representative waveform is an ISO point or a J point indicated in the electrocardiogram waveform shown in FIG. 16. The ISO point is a potential at a flat point of a QRS start portion (i.e., a start point of a Q wave) and the J point is a potential at an inflection point of a curve that goes from an S wave to a T wave.

Then the ST measuring unit 130 measures a shape of each representative waveform (S620). A shape of the representative waveform is not determined qualitatively, that is, it is represented by feature values determined quantitatively at individual portions of the representative waveform such as an STJ value, an ST60 value, and a QRS main peak amplitude indicated in the electrocardiogram waveform shown in FIGS. 15 and 16. The STJ value is a potential at the J point, the ST60 value is a potential at a point that is 60 nsec after the J point, and the QRS main peak amplitude is a potential at a highest point of the Q wave, the R wave, and the S wave with respect to a base line.

Then the ST measuring unit 130 judges whether each representative waveform is of ventricular pacing (S630). If the representative waveform is of ventricular pacing (S630: yes), the ST measuring unit 130 makes judgment according to Sgarbossa criteria (for each lead) (S640) and judgment according to Smith criteria (for each lead) (S650) and outputs the representative waveform, the measurement reference point, the STJ value, the ratio (STJ change amount)/(QRS main peak amplitude), whether the Sgarbossa criteria are satisfied, and whether the Smith criteria are satisfied to the analyzing unit 140 (for each lead) (S660).

The ST measuring unit 130 calculates a Sgarbossa total score and a Smith total score for all of the leads (S670) and outputs them (S680). The judgment according to the Sgarbossa criteria and the judgment according to the Smith criteria will be described later.

On the other hand, if an electrocardiogram waveform is not of ventricular pacing S430: no), the ST measuring unit 130 outputs, for each lead, the representative waveform, the measurement reference point, the STJ value, and the ST60 value to the analyzing unit 140 (S690).

### <Judgment According to Sgarbossa Criteria>

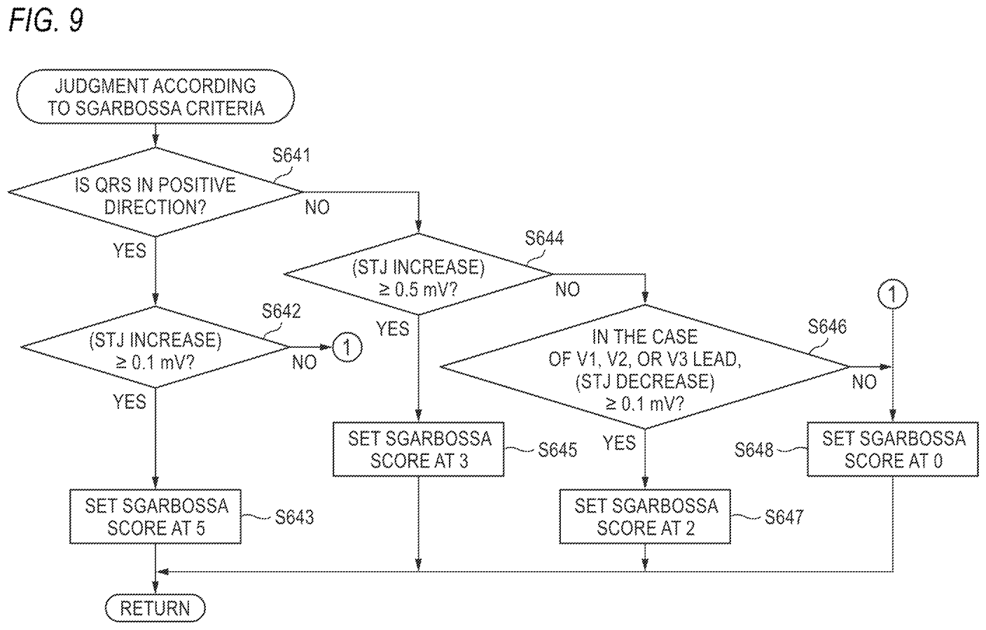

FIG. 9 is a subroutine flowchart of the step <Judgment according to the Sgarbossa criteria> in the flowchart shown in FIG. 1. The subroutine flowchart shown in FIG. 9 is applied to only Specific Embodiments 2 and 3 which assume six electrodes and 10 electrodes, respectively.

The analyzing unit 140 judges whether the QRS of the representative waveform is in the positive direction as shown in FIGS. 15 and 16 (S641), that is, whether the QRS variation direction is upward (positive direction) as shown in FIGS. 15 and 16 or downward.

If the QRS is in the positive direction (S641: yes), then the analyzing unit 140 judges whether the STJ increase is larger than or equal to 0.1 mV (S642). If the STJ increase is larger than or equal to 0.1 mV (S642: yes), the analyzing unit 140 sets the Sgarbossa score at 5 (S643).

If the QRS is not in the positive direction (S641: no), then the analyzing unit 140 judges whether the STJ increase is larger than or equal to 0.5 mV (S642). If the STJ increase is larger than or equal to 0.5 mV (S642: yes), the analyzing unit 140 sets the Sgarbossa score at 3 (S645).

If the STJ increase is smaller than 0.5 mV (S644: no), in the case of a V1, V2, or V3 lead, the analyzing unit 140 judges whether the STJ increase is larger than or equal to 0.1 mV (S646). Like Sgarbossa Smith judgment, threshold values shown in FIG. 22, a setting range of the STJ decrease in the case of a V1, V2, or V3 lead is set to a prescribed range by the manipulation unit 170 (see FIG. 1). The setting range is from 0.10 mV to 1.0 mV (initial value: 0.1 mV, interval: 0.01 mV). Alternatively, a setting range can be set in the form of a distance in mm on a graph of the electrocardiogram waveform rather than voltages. In this case, a setting range is set so as to be from 1.0 mm to 10.0 mm (initial value: 1.0 mm, interval: 0.1 mm). If the STJ decrease is larger than or equal to 0.1 mV (S646: yes), the analyzing unit 140 sets the Sgarbossa score at 2 (S647).

If the STJ increase is smaller than 0.1 mV (S642: no) or if the STJ decrease in the case of a V1, V2, or V3 lead is smaller than 0.1 mV (S646: no), the analyzing unit 140 sets the Sgarbossa score at 0 (S648).

### <Judgment According to Smith Criteria>

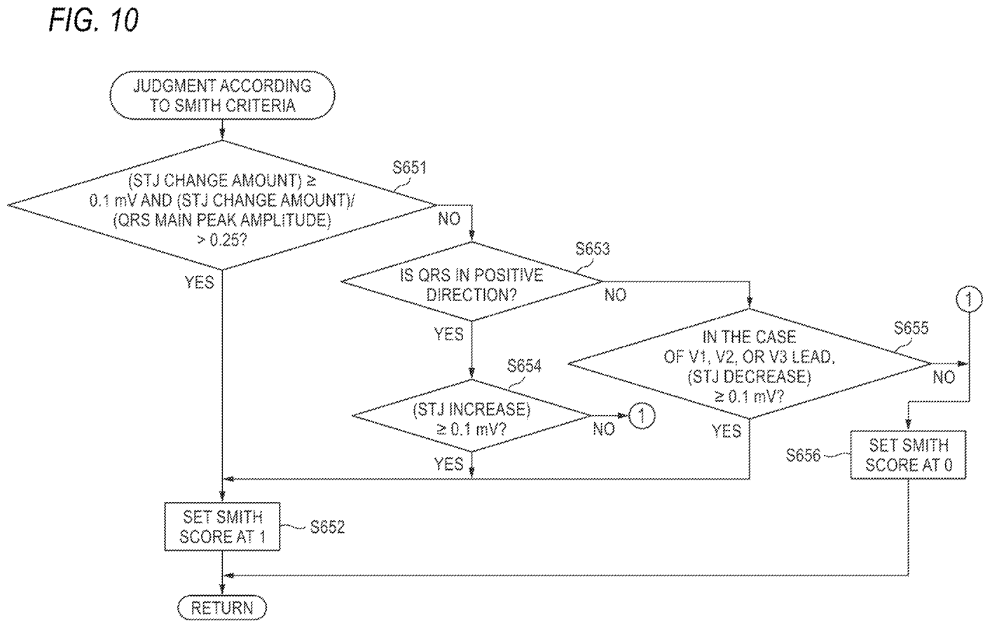

FIG. 10 is a subroutine flowchart of the step <judgement according to the Smith criteria> in the flowchart shown in FIG. 8. The subroutine flowchart shown in FIG. 10 is applied to only Specific Embodiments 2 and 3 which assume six electrodes and 10 electrodes, respectively.

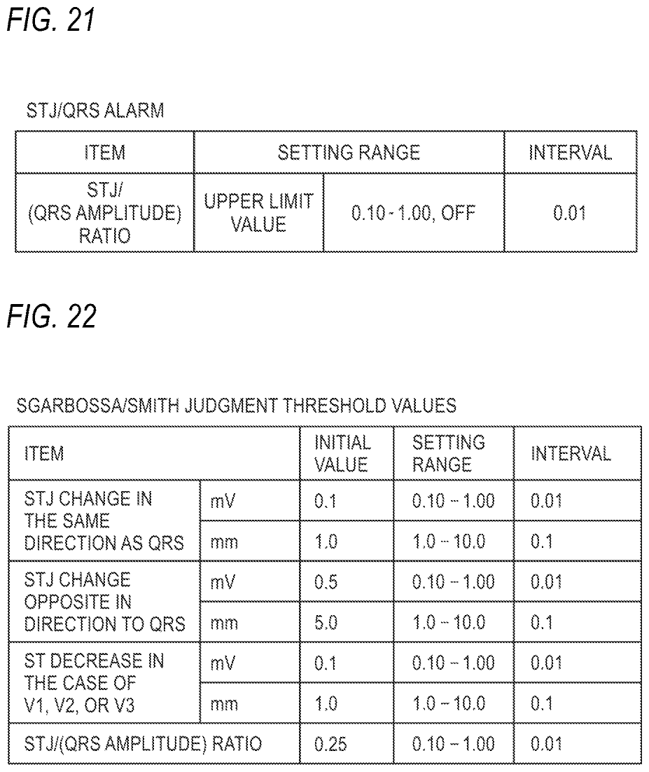

The analyzing unit 140 judges whether the STJ change amount (see FIGS. 15 and 16) is larger than or equal to 0.1 mV and the ratio (STJ change amount)/(QRS main peak amplitude) (see FIGS. 15 and 16) exceeds 0.25 (S651). Like an STJ/QRS alarm shown in FIG. 21, an upper limit of the ratio (STJ change amount)/(QRS main peak amplitude) can be set in a range of 0.10 to 1.00 (interval 0.01). Or the STJ/QRS alarm can be made off. If the STJ change amount is larger than or equal to 0.1 mV and the ratio (STJ change amount)/(QRS main peak amplitude) exceeds 0.25 (S651: yes), the Smith score is set at 1 (S652).

On the other hand, if the STJ change amount is smaller than 0.1 mV or the ratio (STJ change amount)/(QRS main peak amplitude) does not exceed 0.25 (S651: no), the analyzing unit 140 judges whether the QRS in the positive direction (S653). That is, the analyzing unit 140 judges whether the QRS variation direction is upward (positive direction) as shown in FIGS. 15 and 16 or downward.

If the QRS is in the positive direction (S653: yes), then the analyzing unit 140 judges whether the STJ increase is larger than or equal to 0.1 mV (S654). If the STD increase is larger than or equal to 0.1 mV (S654: yes), the analyzing unit 140 sets the Smith score at 1 (S652).

On the other hand, if the QRS is not in the positive direction (S653: no), then the analyzing unit 140 judges whether the STJ decrease is lager than or equal to 0.1 mV (S655). If the STJ decrease is larger than or equal to 0.1 mV (S655: yes), the analyzing unit 140 sets the Smith score at 1 (S652).

If the STJ increase is smaller than 0.1 mV (S654: no) or if the STJ decrease in the case of a V1, V2, or V3 lead is smaller than 0.1 mV (S655: no), the analyzing unit 140 sets the Smith score at 0 (S656).

### <Judgment as to Whether the Notification Conditions are Satisfied>

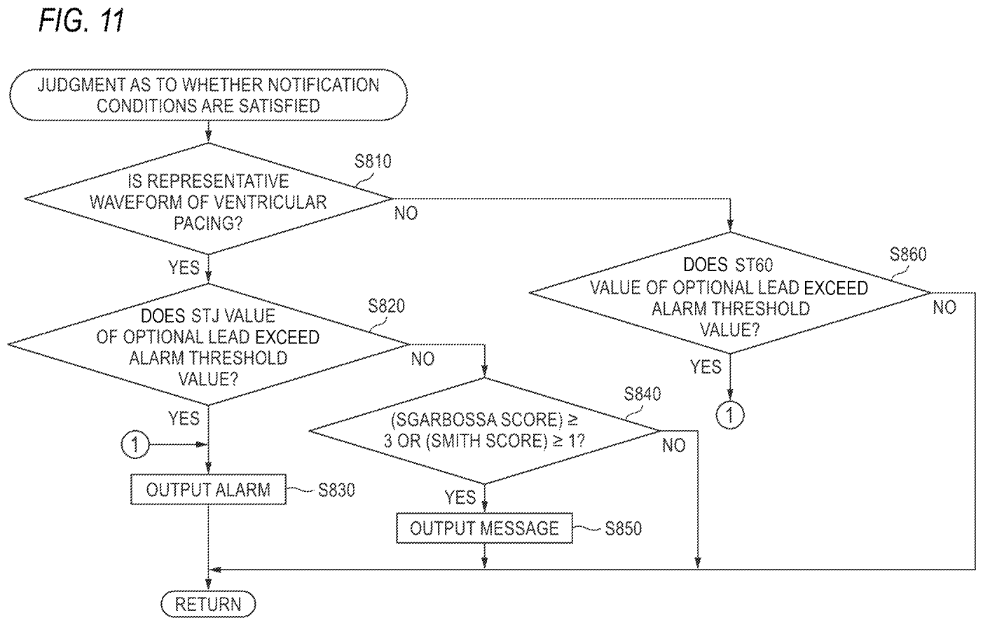

FIG. 11 is a subroutine flowchart of the step <judgement as to whether the notification conditions are satisfied> in the flowchart shown in FIG. 2. The subroutine flowchart shown in FIG. 11 is applied to only Specific Embodiment 2 which assumes six electrodes.

The ST measuring unit 130 judges whether a representative waveform generated in the subroutine flowchart of the step <generation of a representative waveform> (see FIG. 3) is of ventricular pacing (S810). If the representative waveform is of ventricular pacing (S810: yes), then the analyzing unit 140 judges whether the STJ value of an arbitrary lead exceeds an alarm threshold value (S820).

As shown in FIG. 20, an upper limit value and a lower limit value of an alarm relating to the STJ value can each be set in a prescribed range by the manipulation unit 170 (see FIG. 11). The interval of a setting range is 0.01 mV and its upper limit value is from −1.99 mV to +2.00 mV and its lower limit value is from −2.00 mV to +1.99 mV. The setting of the upper limit value and the lower limit value can be made off. Alternatively, a setting range can be set in the form of a distance in mm on a graph of the electrocardiogram waveform rather than voltages. In this case, the interval of a setting range is 0.1 mm and its upper limit value is from −19.99 mm to +20.0 mm and its lower limit value is from −20.0 mm to +19.99 mm. The setting of the upper limit value and the lower limit value can be made off.

If the STJ value of the arbitrary lead exceeds the alarm threshold value (S820: yes), the notification unit 150 outputs an alarm (S830). On the other hand, if the representative waveform is not of ventricular pacing (S810: no), then the analyzing unit 140 judges whether the ST60 value of the arbitrary lead exceeds an alarm threshold value (S860). As shown in FIG. 19, an upper limit value and a lower limit value of an alarm relating, to the ST60 value can each be set in a prescribed range by the manipulation unit 170 (see FIG. 1). The interval of a setting range is 0.01 mV and its upper limit value is from −1.99 mV to +2.00 mV and its lower limit value is from −2,400 mV to +1.99 mV. The setting of the upper limit value and the lower limit value can be made off. Alternatively, a setting range can be set in the form of a distance in mm on a graph of the electrocardiogram waveform rather than voltages. In this case, the interval of a setting range is 0.1 mm and its upper limit value is from −19.9 mm to +20.0 mm and its lower limit value is from −20.0 mm to +19.9 mm. The setting of the upper limit value and the lower limit value can be made off.

If it is judged that the ST60 value of the arbitrary lead exceeds the alarm threshold value (S860: yes), the notification unit 150 outputs an alarm (S830). On the other hand, if the STJ value of the arbitrary lead does not exceed the alarm threshold value (S820: no), then the analyzing unit 140 judges whether the Sgarbossa score is larger than or equal to 3 or the Smith score is larger than or equal to 1 (S840). If the Sgarbossa score is larger than or equal to 3 or the Smith score is larger than or equal to 1 (S840: yes), the notification unit 150 outputs a message (S850).

If it is judged at step S860 that the ST60 value of the arbitrary lead does not exceed the alarm threshold value (S860: no) or it is judged at step S840 that the Sgarbossa score is smaller than 3 and the Smith criteria is smaller than 1 (S840: no), the process is finished without issuing any notice.

How the physiological information processing instrument 100 according to Specific Embodiment 2 operates according to each of the subroutine flowcharts has been outlined above. In summary, the physiological information processing instrument 100 according to Specific Embodiment 2 operates as follows. Electrocardiogram waveforms of a subject person are measured and stored until lapse of a prescribed time. The stored electrocardiogram waveforms are classified for each lead into groups of at least normal beats, atrial pacing beats, and ventricular pacing beats, and a representative waveform is generated. An ST measurement is performed on an electrocardiogram waveform generated for each lead and an ST recall is registered. An alarm or a message is issued if notification conditions are satisfied. This process makes it possible to perform myocardial ischemia monitoring on electrocardiogram waveforms of ventricular pacing that has been difficult to perform in the art. It is noted that even in Specific Embodiment 2 left bundle branch block judgment can be made in the case where chest leads and V1 and V2.

### Specific Embodiment 3

### (Configuration of Physiological Information Processing Instrument)

The configuration of the physiological information processing instrument according to this Specific Embodiment is the same as shown in FIG. 1 (block diagram).

### (Operation of Physiological Information Processing Instrument)

The physiological information processing instrument 100 according to this Specific Embodiment operates, in outline, according to the same procedure as shown in the main flowchart shown in FIG. 2. However, in this Specific Embodiment, since the number of electrodes attached to a subject person is 10 that is different than in Specific Embodiments 1 and 2 and the number of electrocardiogram waveforms obtained is 12, in the main flowchart of FIG. 2 12-lead electrocardiogram waveforms are measured at step S100 and 12-lead electrocardiogram waveforms are stored at step S200. And subroutine flowcharts of step S600 <ST measurement> and step S800 <notification conditions satisfied?> are somewhat different than in Specific Embodiment 1. The procedures of these subroutine flowcharts will be described below.

### <ST Measurement>

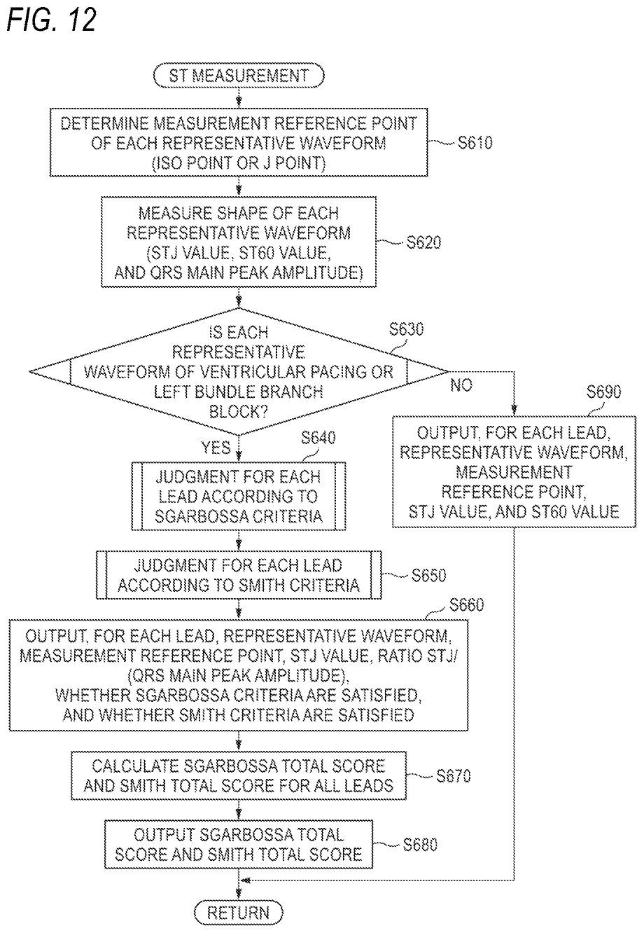

FIG. 12 is a subroutine flowchart of a Specific Embodiment 3 version of the step <ST measurement> in the flowchart of FIG. 2. The subroutine flowchart shown in FIG. 12 is applied to only Specific Embodiment 3 which employs 10 electrodes.

The ST measuring unit 130 determines a measurement reference point of each representative waveform that was generated in the subroutine flowchart <generation of a representative waveform> (see FIG. 3) (S610) In this Specific Embodiment, representative waveforms are generated for the respective leads of 12-lead electrocardiogram waveforms. The measurement reference point of each representative waveform is an ISO point or a J point indicated in the electrocardiogram waveform shown in FIG. 16. The ISO point is a potential at a flat point of a QRS start portion (i.e., a start point of a Q wave) and the J point is a potential at an inflection point of a curve that goes from an S wave to a T wave.

Then the ST measuring unit 130 measures a shape of each representative waveform (S620). A shape of the representative waveform is not determined qualitatively, that is, it is represented by feature values determined quantitatively at individual portions of the representative waveform such as an STJ value, an ST60 value, and a QRS main peak amplitude indicated in the electrocardiogram waveform Shown in FIGS. 15 and 16. The STJ value is a potential at the J point, the ST60 value is a potential at a point that is 60 msec after the 3 point, and the QRS main peak amplitude is a potential at a highest point of the Q wave, the R wave, and the S wave with respect to a base line.

Then the ST measuring unit 130 judges whether each representative waveform is of ventricular pacing or left bundle branch block (S301). If the representative waveform is of ventricular pacing or left bundle branch block (S630: yes), the ST measuring unit 130 makes judgment according to Sgarbossa criteria (for each lead) (S640) and judgment according to Smith criteria (for each lead)(S650) and outputs the representative waveform, the measurement reference point, the STJ value, the ratio (STJ change amount)/(QRS main peak amplitude), whether the Sgarbossa criteria are satisfied, and whether the Smith criteria are satisfied to the analyzing unit 140 (for each lead) (S660).

The ST measuring unit 130 calculates a Sgarbossa total score and a Smith total score for all of the leads (S670) and outputs them (S680). The manners of the judgment according to the Sgarbossa criteria and the judgment according to the Smith criteria are the same as illustrated in the subroutine flowcharts shown in FIGS. 9 and 10.

On the other hand, if an electrocardiogram waveform is not of ventricular pacing or left bundle branch block (S630: no), the ST measuring unit 130 outputs, for each lead, the representative waveform, the measurement reference point, the STJ value, and the ST60 value to the analyzing unit 140 (S690).

### <Judgment According to Sgarbossa Criteria>

The subroutine flowchart of the step <judgment according to Sgarbossa criteria> of this Specific Embodiment in the flowchart of FIG. 12 is the same as shown in FIG. 9.

### <Judgment According to Smith Criteria>

The subroutine flowchart of the step <judgment according to Smith criteria> in the flowchart of FIG. 12 is the same as shown in FIG. 10.

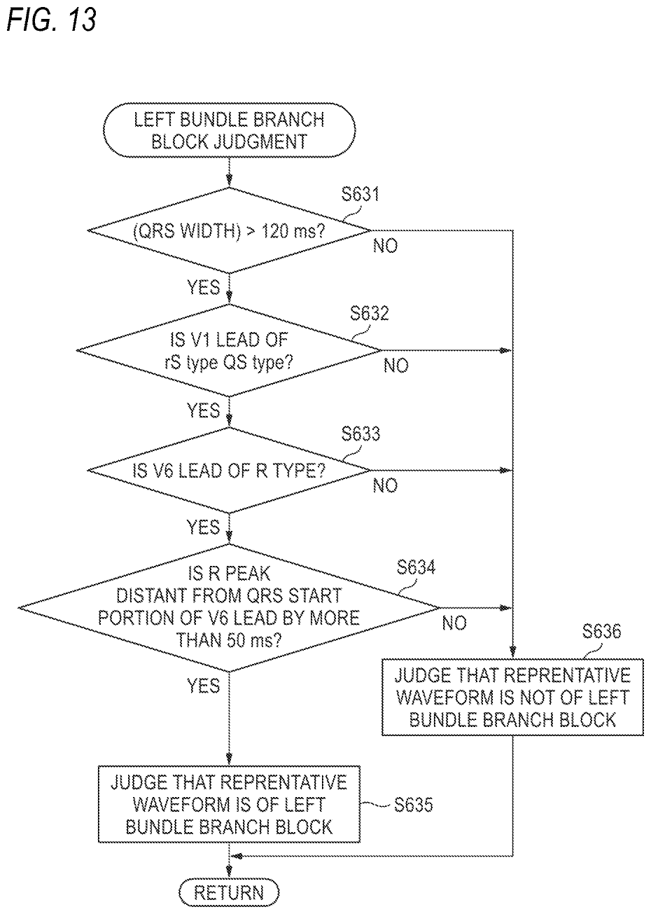

FIG. 13 is a subroutine flowchart of <left bundle branch block judgment> in the step <is each representative waveform of ventricular pacing or left bundle branch block?> in the flowchart of FIG. 12.

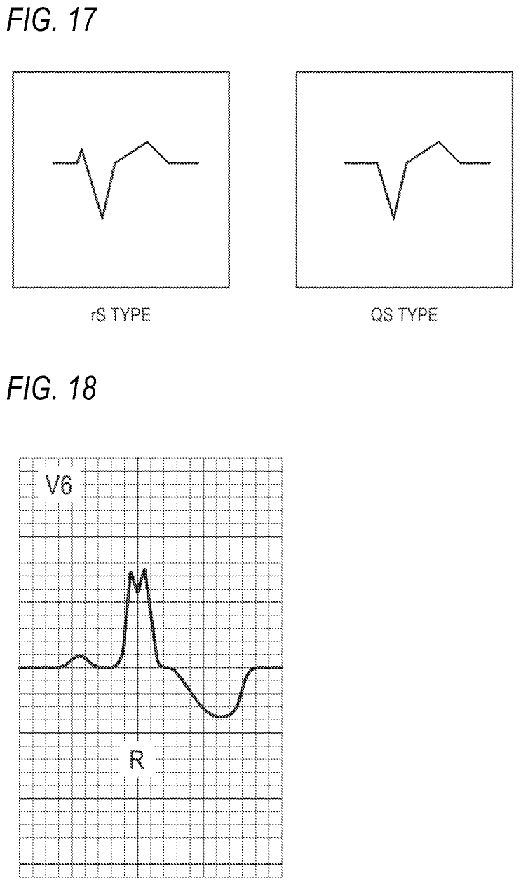

The ST measuring unit 130 judges whether a QRS width as shown in FIG. 15 is larger than 120 msec (S631). If the QRS width is larger than 120 msec (S631: yes), the ST measuring unit 130 judges whether the V1 lead is of an rS type or a QS type (S632). The V1 lead is of the rS type if the electrocardiogram waveform is as shown in the left part of FIG. 17 and the V1 lead is of the QS type if the electrocardiogram waveform is as shown in the right part of FIG. 17. If the V1 lead is of the rS type or the QS type (S632: yes), then the ST measuring unit 130 judges whether the V6 lead is of an R type (S633). The V6 lead being of the R type if the electrocardiogram waveform is as shown in the left part of FIG. 15. If the V6 lead is of the R type (S633: yes), then the ST measuring unit 130 judges whether an R peak is distant from the QRS start portion of the V6 lead by more than 50 msec (S634). If the R peak of the V6 lead is distant from its QRS start portion by more than 50 msec (S634: yes), the ST measuring unit 130 judges that the representative waveform is of left bundle branch block (S635).

On the other hand, if the QRS width is not larger than 120 msec (S631: no), the V1 lead is not of the rS type or the QS type (6632: no), the V6 lead is not of the R type (S633: no), or the R peak of the V6 lead is not distant from its QRS start portion by more than 50 msec (S634: no), the ST measuring unit 130 judges that the representative waveform is not of left bundle branch block (S636).

### <Judgment as to Whether the Notification Conditions are Satisfied>

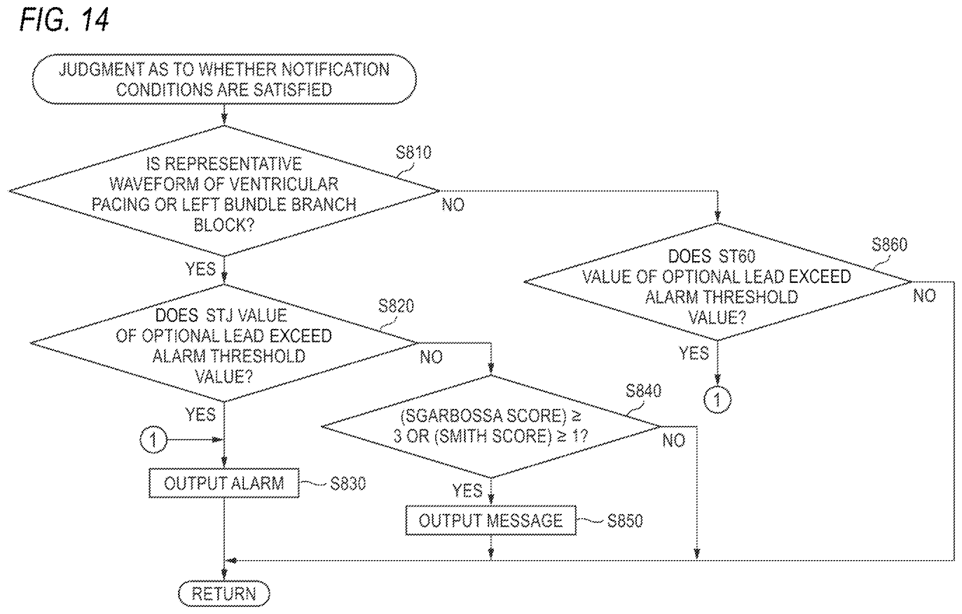

FIG. 14 is a subroutine flowchart of a Specific Embodiment 3 version of the step <judgment as to whether the notification conditions are satisfied> in the flowchart shown in FIG. 2.

The ST measuring unit 130 judges whether a representative waveform generated in the subroutine flowchart of the step <generation of a representative waveform> (see FIG. 3 is of ventricular pacing or left bundle branch block (S810). If the representative waveform is of ventricular pacing or left bundle branch block (S810: yes), then the analyzing unit 140 judges whether the STJ value of an arbitrary lead exceeds an alarm threshold value (S820).

As shown in FIG. 20, an upper limit value and a lower limit value of an alarm relating to the STJ value can each be set in a prescribed range by the manipulation unit 170 (see FIG. 1). The interval of a setting range is 0.01 mV and its upper limit value is from −1.99 mV to +2.00 mV and its lower limit value is from −2.00 mV to +1.99 mV. The setting of the upper limit value and the lower limit value can be made off. Alternatively, a setting range can be set in the form of a distance in mm on a graph of the electrocardiogram waveform rather than voltages. In this case, the interval of a setting range is 0.1 mm and its upper limit value is from −19.99 mm to +20.0 mm and its lower limit value is from −20.0 mm to +19.99 mm. The setting of the upper limit value and the lower limit value can be made off.

If the STJ value of the arbitrary lead exceeds the alarm threshold value (S820: yes), the notification unit 150 outputs an alarm (S830). On the other hand, if the representative waveform is not of ventricular pacing or left bundle branch block (6910: no), then the analyzing unit 141) judges whether the ST60 value of the arbitrary lead exceeds an alarm threshold value (S860). As shown in FIG. 19, an upper limit value and a lower limit value of an alarm relating to the ST60 value can each be set in a prescribed range by the manipulation unit 170 (see FIG. 1). The interval of a setting range is 0.01 mV and its upper limit value is from −1.99 mV to +2.00 mV and its lower limit value is from −2.00 mV to +1.99 mV. The setting of the upper limit value and the lower limit value can be made off. Alternatively, a setting range can beset in the form of a distance in mm on a graph of the electrocardiogram waveform rather than voltages. In this case, the interval of a setting range is 0.1 mm and its upper limit value is from −19.9 mm to +20.0 mm and its lower limit value is from −20.0 mm to +19.9 mm. The setting of the upper limit value and the lower limit value can be made off.

If it is judged that the ST60 value of the arbitrary lead exceeds the alarm threshold value (S860: yes), the notification unit 150 outputs an alarm (S830). On the other hand, if the STJ value of the arbitrary lead does not exceed the alarm threshold value (S820: no), then the analyzing unit 140 judges whether the Sgarbossa score is larger than or equal to 3 or the Smith score is larger than or equal to 1 (S840). If the Sgarbossa score is larger than or equal to 3 or the Smith score is larger than or equal to 1 (S840: yes), the notification unit 150 outputs a message (S850).

If it is judged at step S860) that the ST60 value of the arbitrary lead does not exceed the alarm threshold value (S860: no) or it is judged at step S840 that the Sgarbossa score is smaller than 3 and the Smith criteria is smaller than 1 (S840: no), the process is finished without issuing any notice,

How the physiological information processing instrument 100 according to Specific Embodiment 3 operates according to each of the subroutine flowcharts has been outlined above. In summary, the physiological information processing instrument 100 according to Specific Embodiment 3 operates as follows. Electrocardiogram waveforms of a subject person are measured and stored until lapse of a prescribed time. The stored electrocardiogram waveforms are classified for each lead into groups of at least normal beats, atrial pacing beats, ventricular pacing beats, and left bundle branch block beats, and a representative waveform is generated. An ST measurement is performed on an electrocardiogram waveform generated for each lead and an ST recall is registered. An alarm or a message is issued if notification conditions are satisfied. This process makes it possible to perform myocardial ischemia monitoring on electrocardiogram waveforms of ventricular pacing or left bundle branch block that has been difficult to perform in the art.

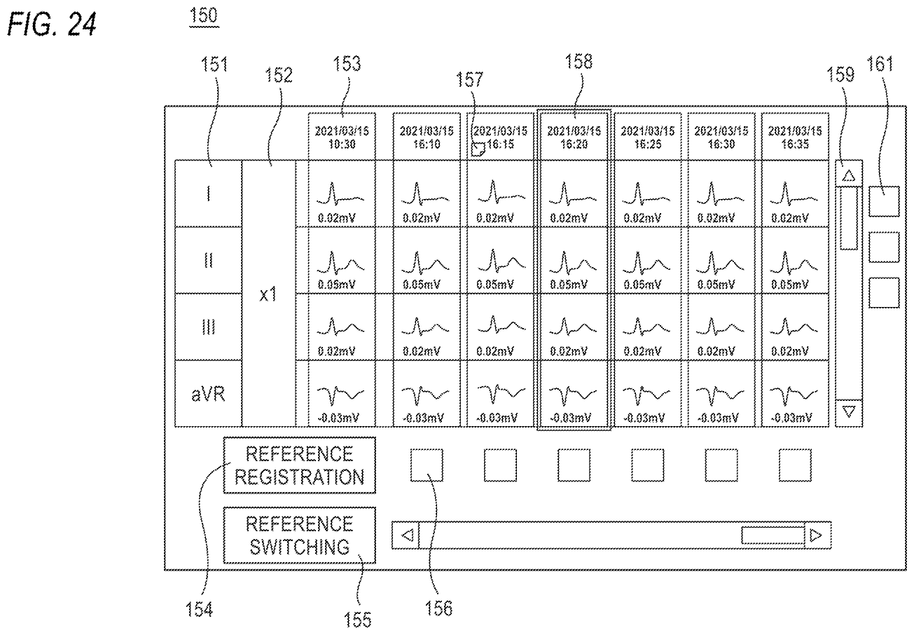

Next, how the physiological information processing instrument 100 according to the presently disclosed subject matter issues an alarm or a message relating to myocardial ischemia. An alarm or a message relating to myocardial ischemia is issued by the notification unit 150 (see FIG. 1) auditorily or visually. Specific example manners of visual notification by the notification unit 150, that is, specific example manners of display, are shown in FIGS. 23 and 24. The specific example manners of display shown in these drawings are common to Specific Embodiments 1-3. These specific example manners of display will be described below.

FIG. 23 shows specific examples of an ST value and an ST recall waveform. As shown in FIG. 23, the notification unit 150 displays on its screen, as an analysis result of an ST measurement, a representative waveform generated by the judging unit 120 and a measurement value (ST value 0.05 mV) relating to a shape of an electrocardiogram waveform.

In the case of ventricular pacing and left bundle branch block, since the ST value is interpreted differently than in an ordinary case, the notification unit 150 displays an ST recall in a modified manner. More specifically, the frame line of an ST recall waveform is graded according to the ST value; usually, the color is simply made deeper as the ST value goes away from 0. That is, the color of the frame line of the ST recall waveform shown in FIG. 23 is made deeper as the ST value becomes larger. Instead of making the frame line deeper, the frame line may be made thicker or its color may be changed. In the case of ventricular pacing and left bundle branch block, a variation in the direction opposite to QRS may be regarded as being 0 until the ST value exceeds 0.5 mV (or ¼ of a QRS amplitude) and color gradation may be started after the ST value exceeds 0.5 mV (or ¼ of the QRS amplitude).

FIG. 24 is a table for description of display forms of an ST recall. As shown in FIG. 24, the notification unit 150 is configured so as to be able to display analysis results of an ST measurement on its screen in various forms. For example, the notification unit ISO is provided with a function of comparing a waveform obtained at an arbitrary time with a waveform registered as a reference. That is, the notification unit 150 is configured so that two kinds of references, that is a reference for normal beats and a reference for ventricular pacing or a reference for normal beats and a reference for left bundle branch block, can be registered therein to enable selection between them depending on which of them a user wants to see

Thus, the notification unit 150 has a function of displaying, on the screen, an electrocardiogram waveform of a normal beat or an electrocardiogram waveform of a ventricular pacing beat or a left bundle branch block beat in such a manner that switching can be made between them. The notification unit 150 also has a function of registering an electrocardiogram waveform of a normal beat and an electrocardiogram waveform of a ventricular pacing beat or a left bundle branch block beat

As shown in FIG. 24, a picture of the notification unit 150 has lead name display boxes 151, sensitivity display boxes 152, a reference display box 153, a reference registration portion 154, a reference switching portion 155, check boxes 156, a comment display mark 157, a cursor frame 158, a scroll portion 159), and display manipulation portions 161.

Each lead name display box 151 is for display of a lead of an ST recall that is displayed on the right of this box. For example, in Specific Embodiment 1, since three electrodes are attached to a target person, only information of one lead is displayed. In Specific Embodiment 2 or 3, since 6 or 10 electrodes are attached to a target person, information of eight or 12 leads is displayed. Although information of only four leads is displayed in FIG. 24, information of other leads can be displayed by sliding the scroll portion 159. In the case of eight leads, information of the other four leads that is not displayed in FIG. 24 can be displayed by sliding the scroll portion 159. In the case of 12 leads, information of the other eight leads that is not displayed in FIG. 24 can be displayed by sliding the scroll portion 159. Information of a lead to be displayed can be changed by touching the lead name shown in a lead name display box 151.

ST recalls of each lead are displayed in time series so as to move left to right every predetermined time (e.g., every five minutes). In FIG. 24, ST recalls registered in 25 minutes are displayed in time series.

The display sensitivity can be changed by touching a sensitivity display box 152. In FIG. 24, the display sensitivity is set at 1.

Normal beats or ventricular pacing beats that have been registered by manipulating the reference registration portion 154 are displayed in the reference display box 153. The presence of the reference display box 153 makes it easier to compare ST recall waveforms display on the right of this box in time series with each normal beat or ventricular pacing beat displayed in the reference display box 153.

ST recalls selected by the cursor frame 15K are registered as references by manipulating the reference registration portion 154.

The reference switching portion 155 is to select (switch) to one a user wants to see among plural kinds of references that have been registered by manipulating the reference registration portion 154.

The check boxes 156 are used to select a file that a user wants to record or print. The comment display mark 157 is displayed when a comment has been input. The cursor frame 158 is used to specify a file that a user wants to register as a reference. The scroll portion 159 serves to display ST recalls of a lead that are not displayed in the picture when it is slide-manipulated. The display manipulation portions 161 serve to switch the picture to divisional display, full-screen display, or some other review picture or to set a display time interval of ST recalls.

The physiological information processing instrument, the physiological information processing method, and the control program of a physiological information processing instrument according to the presently disclosed subject, matter have been described above in detail on the basis of the embodiment. However, technical scopes of the physiological information processing method, and the control program of a physiological information processing instrument according to the presently disclosed subject matter are not limited to the contents of the above-described embodiment.

1. A physiological information processing instrument comprising:
a memory that stores instructions; and
a processor that executes the instructions stored in the memory to:
judge whether an electrocardiogram waveform of a subject person is of at least one of a ventricular pacing beat and a left bundle branch block beat;
perform an ST measurement by applying myocardial ischemia evaluation criteria to the electrocardiogram waveform that has been judged of at least one of a ventricular pacing beat and a left bundle branch block beat; and
analyze results of the ST measurement and outputting an analysis result and information relating, to myocardial ischemia in the form of audible or visible information.

2. The physiological information processing instrument according to claim 1, further comprising a notifier which announces the analysis result and information relating to myocardial ischemia in the form of an alarm or a message.

3. The physiological information processing instrument according to claim 1, wherein the processor classifies part of electrocardiogram waveforms of the subject person as electrocardiogram waveforms of ventricular pacing beats or left bundle branch block beats or both, generates a ventricular pacing beat group or a left bundle branch block beat group, and judges whether each electrocardiogram waveform is an electrocardiogram waveform of at least one of a ventricular pacing beat and a left bundle branch block beat using a representative waveform generated from the ventricular pacing beat group or the left bundle branch block beat group.

4. The physiological information processing instrument according to claim 3, wherein the processor employs, as a representative waveform of a ventricular pacing beat, an average waveform generated plural electrocardiogram waveforms belonging to the ventricular pacing beat group or employs, as a representative waveform of a left bundle branch block beat, an average waveform generated plural electrocardiogram waveforms belonging to the left bundle branch block beat group.

5. The physiological information processing instrument according to claim 1, wherein the processor judges whether each electrocardiogram waveform is a ventricular pacing beat in the case where the number of electrodes attached to the subject person is three or six, and judges whether each electrocardiogram waveform is a ventricular pacing beat or a left bundle branch block beat in the case where the number of electrodes attached to the subject person is 10.

6. The physiological information processing instrument according to claim 1, wherein processor performs the ST measurement by applying, as the myocardial ischemia evaluation criteria, Sgarbossa criteria or Smith criteria or both and measurement values relating to a shape of an electrocardiogram waveform that has been judged to be a ventricular pacing beat or measurement values relating to a shape of an electrocardiogram waveform that has been judged to be a left bundle branch block beat.

7. The physiological information processing instrument according to claim 6, wherein the processor performs judgment according to Sgarbossa criteria and Smith criteria by judging whether an electrocardiogram waveform judged of a ventricular pacing beat satisfies the Sgarbossa criteria and the Smith criteria in the case where the number of electrodes attached to the subject person is three, performs judgment according to Sgarbossa criteria and Smith criteria depending on scores, determined according to Sgarbossa criteria and Smith criteria that are different than employed in the case of the number of electrodes being equal to three, of an electrocardiogram waveform judged of a ventricular pacing beat in the case where the number of electrodes attached to the subject person is six, and performs judgment according to Sgarbossa criteria and Smith criteria depending on scores, determined according to the same Sgarbossa criteria and Smith criteria as an electrocardiogram waveform is judged of a ventricular pacing beat in the case of the number of electrodes being equal to six, of an electrocardiogram waveform judged of a left bundle branch block beat in the case where the number of electrodes attached to the subject person is 10.

8. The physiological information processing instrument according to claim 7, wherein in the case where the number of electrodes attached to the subject person is six or 10, the processor determines scores of the Sgarbossa criteria and the Smith criteria of an electrocardiogram waveform of a ventricular pacing beat or a left bundle branch block beat of each lead and performs judgment according to the Sgarbossa criteria and the Smith criteria by calculating a total score of all leads.

9. The physiological information processing instrument according to claim 8, wherein the processor uses, as measurement values relating to a shape of an electrocardiogram waveform used in performing the ST measurement, at least measurement reference points (ISO point and J point), an STJ value, and (STJ change amount)/(QRA main peak amplitude) ratio of the electrocardiogram waveform.

10. The physiological information processing instrument according to claim 9, wherein the processor outputs an alarm if the STJ value exceeds a set, alarm threshold, and outputs a message if the Sgarbossa criteria or the Smith criteria are satisfied even if the STJ value does not exceed the set alarm threshold.

11. The physiological information processing instrument according to claim 9, wherein the processor outputs an alarm if an STJ value of an arbitrary lead exceeds a set alarm threshold, and outputs a message if a total score of the Sgarbossa criteria and the Smith criteria exceeds a set total score even if the STJ value of the arbitrary lead does not exceed a set alarm threshold.

12. The physiological information processing instrument according to claim 3, wherein the notifier displays, on a screen, the representative waveform generated by the processor and measurement values relating to a shape of an electrocardiogram waveform.

13. The physiological information processing instrument according to claim 12, wherein the notifier has a function of displaying, on a screen, an electrocardiogram waveform of a normal beat or an electrocardiogram waveform of a ventricular pacing beat or a left bundle branch block beat in such a manner that switching can be made between them selectively.

14. The physiological information processing instrument according to claim 13, wherein the notifier has a function of registering an electrocardiogram waveform of a normal beat or an electrocardiogram waveform of a ventricular pacing beat or a left bundle branch block beat.

15. The physiological information processing instrument according to claim 14, wherein the notifier displays a frame line of a displayed electrocardiogram waveform in such a manner that the frame line becomes deeper as an ST value of the electrocardiogram waveform becomes larger.

16. A physiological information processing method which makes it possible to myocardial ischemia monitoring using even an electrocardiogram waveform of ventricular pacing or left bundle branch block, comprising the steps of:
judging whether an electrocardiogram waveform of a subject person is of at least one of a ventricular pacing beat and a left bundle branch block beat;
performing an ST measurement by applying myocardial ischemia evaluation criteria to the electrocardiogram waveform that has been judged of at least one of a ventricular pacing beat and a left bundle branch block beat; and
analyzing results of the ST measurement and outputting an analysis result and information relating to myocardial ischemia in the form of audible or visible information.

17. A non-transitory computer-readable storage medium that stores a control program for a physiological information processing instrument, the control program causing a processor to function as:
a judging unit which Judges whether an electrocardiogram waveform of a subject person is of at least one of a ventricular pacing beat and a left bundle branch block beat;
an ST measuring unit which performs an ST measurement by applying myocardial ischemia evaluation criteria to the electrocardiogram waveform that has been judged of at least one of a ventricular pacing beat and a left bundle branch block beat; and
an analyzing unit which analyzes results of the ST measurement and outputting an analysis result and information relating to myocardial ischemia in the form of audible or visible information.
